(19) United States
(12) Patent Application Publication
(10) Pub. No.: US 2024/0324939 A1
(43) Pub. Date: Oct. 3, 2024

(54) ELECTROPHYSIOLOGICAL MAPPING IN THE PRESENCE OF INJURY CURRENT

(71) Applicant: Biosense Webster (Israel) Ltd., Yokneam (IL)

(72) Inventors: Assaf GOVARI, Haifa (IL); Andres Claudio ALTMANN, Haifa (IL); Vadim GLINER, Haifa (IL)

(73) Assignee: Biosense Webster (Israel) Ltd., Yokneam (IL)

(21) Appl. No.: 18/743,766

(22) Filed: Jun. 14, 2024

Related U.S. Application Data
(63) Continuation of application No. 17/492,933, filed on Oct. 4, 2021, now Pat. No. 12,011,280.

(51) Int. Cl. A61B 5/367; A61B 5/00; A61B 5/256; A61B 5/283; A61B 5/339

(52) U.S. Cl. CPC A61B 5/367; A61B 5/256; A61B 5/283; A61B 5/339; A61B 5/7221; A61B 5/7225

(57) ABSTRACT

A system includes an interface and a processor. The interface is configured to receive an electrogram acquired in a heart of a patient. The processor is configured to (i) estimate a level of injury current present in the electrogram, and (ii) based on the estimated level of injury current, decide whether to use the electrogram in a subsequent analysis.

### CROSS-REFERENCE TO RELATED APPLICATIONS

This application is a continuation of prior filed U.S. patent application Ser. No. 17/492,933 (Attorney Docket No.: BIO6579USNP1-253757.000220), filed Oct. 4, 2021, the entire contents of which is hereby incorporated by reference as if set forth in full herein.

### FIELD OF THE INVENTION

The present invention relates generally to processing of electrophysiological signals, and specifically to enabling electrophysiological mapping in the presence of signal baseline wander.

### BACKGROUND OF THE INVENTION

Various methods to account for electrophysiological signal noise have been proposed in the patent literature. For example, U.S. Patent Application Publication 2007/0021679 describes the analysis of surface electrocardiogramd intracardiac signals to identify and separate electrical activity corresponding to distinct but superimposed events in the heart. The analysis assesses the spatial phase, temporal phase, rate, spectrum and reproducibility of each event to determine uniformity of activation in all spatial dimensions. The analysis uses numerical indices derived from these analyses to diagnose arrhythmias. The analysis uses these indices to determine the location of an arrhythmia circuit, and to direct the movement of an electrode catheter to this location for ablation or permanent catheter positioning. The analysis uses variability in these indices from the surface electrocardiogram to indicate subtle beat-to-beat fluctuations which reflect the tendency towards atrial and ventricular arrhythmias. The analysis is generally performed after baseline correction, which makes the analysis insensitive to noise factors including baseline wander.

### SUMMARY OF THE INVENTION

An embodiment of the present invention that is described hereinafter provides a system including an interface and a processor. The interface is configured to receive an electrogram acquired in a heart of a patient. The processor is configured to (i) estimate a level of injury current present in the electrogram, and (ii) based on the estimated level of injury current, decide whether to use the electrogram in a subsequent analysis.

In some embodiments, the processor is configured to use the electrogram when the level of injury current is below a threshold, and to discard the electrogram when the level of injury current is above the threshold.

In some embodiments, the electrogram is a unipolar signal acquired between a reference electrode immersed in blood and a sensing electrode in contact with tissue.

In an embodiment, the processor is configured to estimate the level of injury current by comparing a peak level of the electrogram to a threshold.

In another embodiment, the processor is configured to use the electrogram by annotating the electrogram and using the annotation in an EP map.

In some embodiments, the processor is configured to annotate a local activation time (LAT) value in the electrogram.

There is additionally provided, in accordance with an embodiment of the present invention, a system including an interface and a processor. The interface is configured to receive at least two EP signals acquired in a heart of a patient by a catheter. The processor is configured to (a) using the at least two EP signals, estimate a level of injury current present in an EP signal derived from the at least two EP signals, and (b) based on the estimated level of injury current, decide whether to use the electrogram in a subsequent analysis.

In some embodiments, the processor is configured to derive the EP signal by selecting one of the at least two EP signals.

In some embodiments, one of the at least two EP signals is an electrogram acquired using a reference electrode immersed in blood and a surface electrode, and another of the at least two EP signals is an electrogram acquired using a sensing electrode in contact with tissue and the surface electrode.

In an embodiment, the surface electrode is a WCT terminal.

In another embodiment, the processor is configured to estimate the level of injury current by comparing between at least two of the EP signals.

In some embodiments, the processor is configured to use the derived EP signal by annotating the derived EP signal and using the annotation in an EP map.

In some embodiments, the processor is configured to annotate a local activation time (LAT) value in the derived EP signal.

There is further provided, in accordance with an embodiment of the present invention, a method including receiving an electrogram acquired in a heart of a patient. A level of injury current present in the electrogram is estimated. Based on the estimated level of injury current, it is decided whether to use the electrogram in a subsequent analysis.

There is furthermore provided, in accordance with an embodiment of the present invention, a method including receiving at least two EP signals acquired in a heart of a patient by a catheter. Using the at least two EP signals, a level is estimated, of injury current present in an EP signal derived from the at least two EP signals. Based on the estimated level of injury current, it is decided whether to use the electrogram in a subsequent analysis.

The present invention will be more fully understood from the following detailed description of the embodiments thereof, taken together with the drawings in which:

### BRIEF DESCRIPTION OF THE DRAWINGS

FIG. 1 is a schematic, pictorial illustration of a catheter-based electrophysiological (EP) sensing and signal-analysis system, according to an embodiment of the present invention;

FIG. 2 is a schematic graph of a unipolar electrogram acquired by the system of FIG. 1, which demonstrates a degrading effect of injury current on the electrogram, according to an embodiment of the present invention;

FIG. 3 is a flow chart describing a method to allow for injury current when EP mapping, by detecting and avoiding distorted electrograms, to an embodiment of the present invention;

FIG. 4 is a flow chart describing a method for detecting electrograms distorted by injury current by comparing electrogram peak amplitude to threshold, and dropping such electrograms, according to an embodiment of the present invention; and

FIG. 5 is a flow chart describing a method for detecting electrograms distorted by injury current by comparing baseline signals, according to another embodiment of the present invention.

### DETAILED DESCRIPTION OF EMBODIMENTS

### Overview

Intracardiac electrophysiological (EP) mapping is a catheter-based method that is sometimes applied to characterize cardiac EP wave propagation abnormalities, such as abnormalities that cause arrhythmia. In a typical catheter-based procedure, a distal end of a catheter, which comprises multiple sensing electrodes, is inserted into the heart to sense a set of data points comprising measured locations over a wall tissue of a cardiac chamber and a respective set of EP signals (e.g., intra-cardiac electrograms (EGM)), from which the EP mapping system can produce an EP map of the cardiac chamber. Further to this, available cardiac data may also include multi-channel (e.g., 12-channel) extra-cardiac electrocardiograms (ECG).

During EP signal acquisition, a well-known signal artifact is the presence of “injury current,” which manifests itself as a baseline shift, or baseline wander, due to the presence of very low-frequency components (e.g., DC) in the acquired signal. The injury current is typically caused by the acquiring electrode pushing on cardiac cells, so that the cells, in turn, polarize. It is possible in principle to try and filter-out the injury current by applying a high-pass filter to the signal; however, the filtered signal may still have artifacts that cause errors when the signals are analyzed, such as errors in annotation of late activation time (LAT).

Embodiments of the present invention that are described herein provide methods and systems for detecting and mitigating the effects of injury current in EP mapping procedures. Some disclosed techniques analyze acquired electrograms used for mapping, for detecting the presence of injury current. When injury current is detected in a certain electrogram (also referred to herein simply as “EP signal” or “signal”), e.g., by one of the algorithms described below, the signal is not used for mapping. If there is no detected injury current, the signal is used, for example, by being annotated as an activation in an EP map, such as an LAT map.

In some embodiments, the electrogram signals are acquired using a multi-electrode catheter, such as a basket catheter, which has a central reference electrode. In these implementations, the electrograms are unipolar signals that are measured between the central reference electrode of the basket (which does not contact tissue and is assumed to be a local ground) and a spine electrode in contact with the tissue. In these embodiments, a processor assumes that injury current is present if the acquired signal (typically peak level of the signal) is greater than a preset threshold, for example 50 μV or 100 ρV. Typically, if the peak signal is greater than the threshold, it is not used for mapping.

A multi-electrode catheter with a central reference electrode can be realized in other ways that are consistent with the disclosed technique. For example, a multi-arm catheter nay comprise such a reference electrode disposed on a distal end of a shaft of the catheter, just proximally to the origin point of the arms. Non-limiting examples of such catheters are described in U.S. Patent Applications Publications 2017/0172442 and 2017/0319144, whose disclosures are incorporated herein by reference.

The rationale behind the above-described technique is that, typically, the baseline level changes by injury current are acquired by a spine electrode, but not by the central electrode. In contrast, baseline level changes that are caused by other factors, such as far-field transmission or local environmental conditions (e.g., from scar tissue), are typically acquired by both electrodes.

In an embodiment, the baseline level of the central electrode, V_{CRE}, is measured with respect to a Wilson Central Terminal (WCT) surface electrode configuration described below. (The surface electrodes can also be used to acquire the aforementioned ECGs.) Similarly, the baseline level of a spine electrode, V_{SE}, is measured with respect to the WCT set. If V_{CRE}=V_{SE} (within a given tolerance) then it is assumed that no injury current is present, and the signal may thus be accepted for mapping; if V_{CRE}≠V_{SE} then the presence of injury current is assumed, and the signal is not accepted for mapping.

### System Description

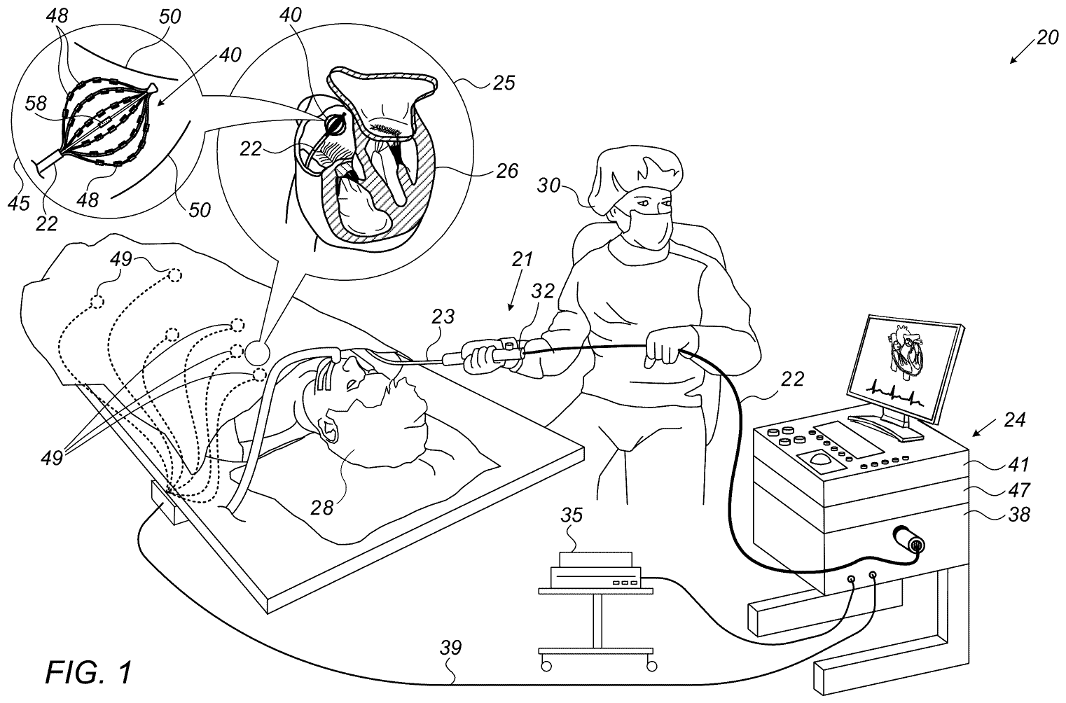

FIG. 1 is a schematic, pictorial illustration of a catheter-based electrophysiological (EP) sensing and signal-analysis system 20, according to an embodiment of the present invention. In the embodiment described herein, catheter 21 is used for EP mapping of heart 26. Further, an ECG recording instrument 35 may receive various types of ECG signals sensed by system 20 during the process.

As shown in inset 25, system 20 comprises a catheter 21 having a multi-electrode basket assembly 40 fitted on a distal end of a shaft 22 of the catheter. Shaft 22 of catheter 21 is navigated by a physician 30 into a heart 26 of a patient 28. Physician 30 inserts shaft 22 through a sheath 23, while manipulating shaft 22 using a manipulator 32 near the proximal end of the catheter.

In an embodiment, basket assembly 40 is configured to perform EP mapping of a cardiac chamber of heart 26 obtaining electrophysiological signals from cardiac chamber surfaces 50. Inset 45 shows an enlarged view of basket catheter 40 inside a cardiac chamber of heart 26. As seen, basket catheter 40 comprises an array of electrodes 48 coupled onto spines that form the basket shape. Basket assembly 40 further includes a central reference electrode 58 which is in contact with a blood pool only. The proximal end of catheter 21 is connected to a control console 24, to transmit, for example, electrograms acquired by electrodes 48.

Using reference electrode 58, basket catheter 40 can acquire unipolar electrograms by measuring potential difference between the central electrode of the basket (which does not contact tissue and is assumed to be a local ground) and a spine electrode 48 in contact with the tissue. As described above, a unipolar electrogram acquired by an electrode 48 that exerts too much force on chamber surface 50 tissue may suffer from injury current (e.g., baseline shift) that degrades the signal. The disclosed technique mitigates degradation of an EP map constructed with some of the raw data (the acquired data points) suffering from injury current, as described above and in method FIG. 3.

Console 24 comprises a processor 41, typically a general-purpose computer, with suitable front end and interface circuits 38 for receiving EP signals (e.g., ECGs and EGMs) as well as non-EP signals (such as position signals) from electrodes 48 of catheter 21. The EP signals are stored in a memory 47 of the processor.

To receive EP signals, processor 41 is connected to electrodes 48 via wires running within shaft 22. Interface circuits 38 are further configured to receive ECG signals, such as from a 12-lead ECG apparatus that can be ECG recording instrument 35, as well as non-ECG signals from surface body electrodes 49. Typically, electrodes 49 are attached to the skin around the chest and legs of patient 28. Processor 41 is connected to electrodes 49 by wires running through a cable 39 to receive signals from electrodes 49.

Four of surface body electrodes 49 are named according to standard ECG protocols: MA (right arm), LA (left arm), ML (right leg), and LL (left leg). A Wilson Central Terminal (WCT) may be formed by three of the four named body surface electrodes 49, and a resulting ECG signal, V_{WCT}, is received by interface circuits 38.

During an EP mapping procedure, the locations of electrodes 48 are tracked while they are inside heart 26 of the patient. For that purpose, electrical signals are passed between electrodes 48 and body surface electrodes 49. Based on the signals, and given the known positions of electrodes 22 on the patient's body, a processor 28 calculates an estimated location of each electrode 22 within the patient's heart. Such tracking may be performed using the Active Current Location (ACL) system, made by Biosense-Webster (Irvine California), which is described in U.S. Pat. No. 8,456,182, whose disclosure is incorporated herein by reference.

The processor may thus associate any given signal received from electrodes 48, such as EGMs, with the location at which the signal was acquired. Processor 41 uses information contained in these signals (e.g., to annotate EP signals) to construct an EP map, such as a local activation time (LAT) map, to present on a display.

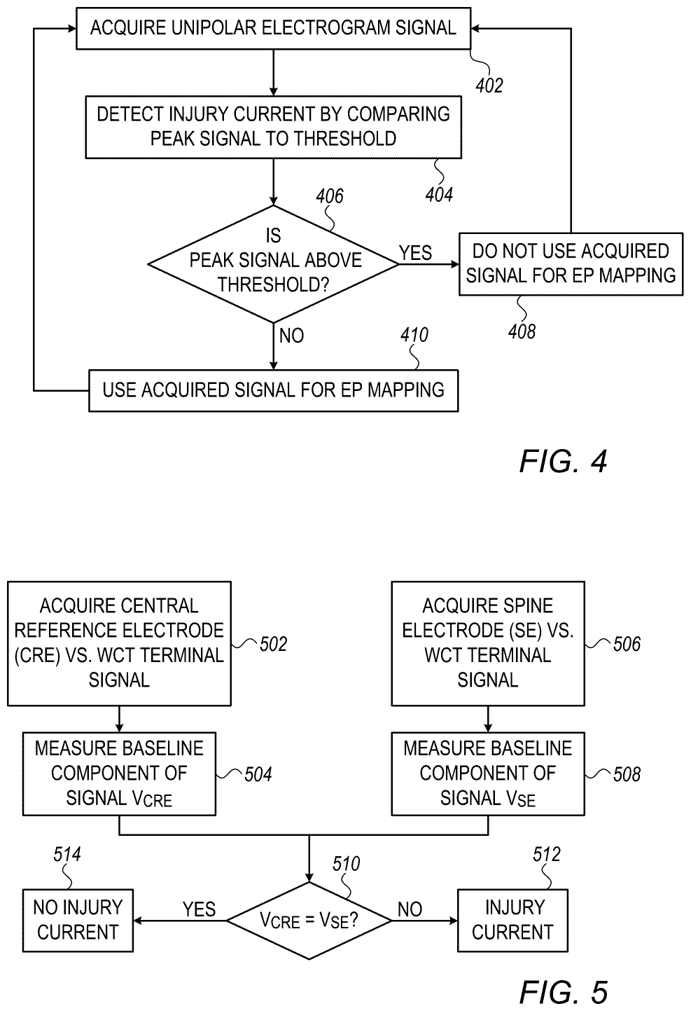

Processor 41 is typically programmed in software to carry out the functions described herein. The software may be downloaded to the processor in electronic form, over a network, for example, or it may, alternatively or additionally, be provided and/or stored on non-transitory tangible media, such as magnetic, optical, or electronic memory. In particular, processor 41 runs a dedicated algorithm as disclosed herein, included in FIGS. 3 and 4, that enables processor 41 to perform the disclosed steps, as further described below.

Although the pictured embodiment in FIG. 1 relates specifically to the use of a basket catheter for cardiac mapping, other distal-end assemblies may be used, such as the aforementioned arcuated Lasso® catheter or the multi-arm Pentaray® catheter. Moreover, the different distal-end assemblies may be further used to perform an electrical ablation.

### Baseline Shift in Cardiac Electrophysiological Signals

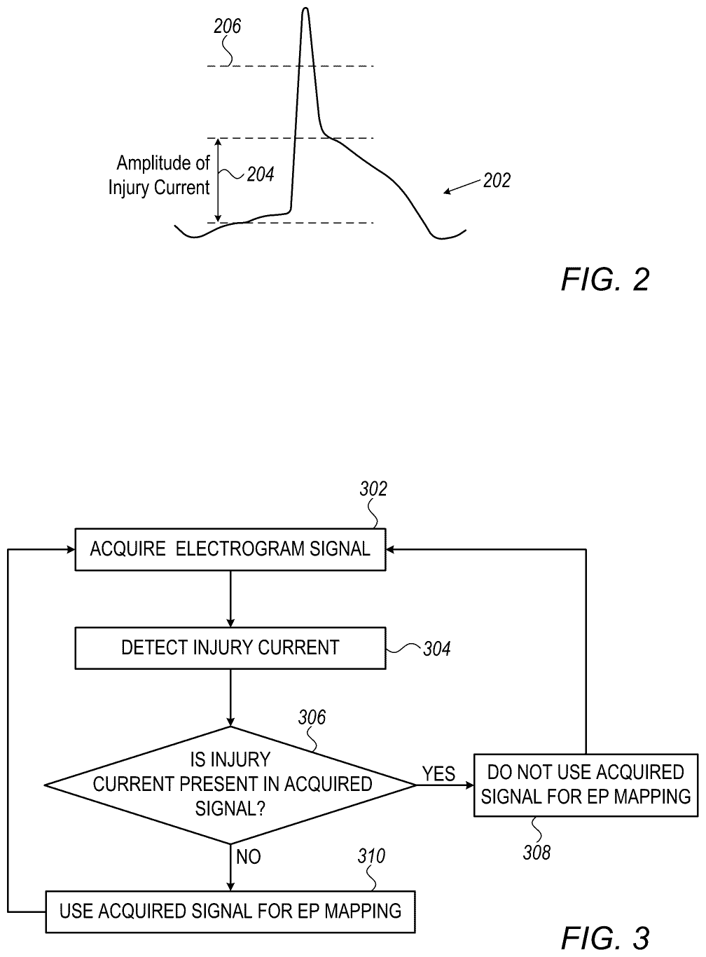

FIG. 2 is a schematic graph of a unipolar electrogram 202 acquired by system 20 of FIG. 1, which demonstrates a degrading effect of injury current on the electrogram, according to an embodiment of the present invention. The plotted waveform is of a potential difference between one of electrodes 48 of catheter assembly 40 and its center reference electrodes 58.

The presence of injury current is manifested as a baseline shift of an amplitude 204 in the unipolar amplitude. This shift causes broadening of the signal. In the shown embodiment, the baseline shift causes the signal to exceed a predefined threshold value 206. This can be used as a criterion to detect and omit from use (e.g., for constructing an EP map) EP signals that were degraded by injury current. For example, a processor can be configured to assume that injury current is present if the acquired EP signal amplitude is greater than the preset threshold 206, for example 50 μV or 100 μV.

### Allowing for Injury Current in Ep Mapping

FIG. 3 is a flow chart describing a method to allow for injury current when EP mapping, by detecting distorted electrograms and avoiding using them, according to an embodiment of the present invention. The algorithm, according to the presented embodiment, carries out a process that begins with interface circuits 38 receiving an electrogram acquired by catheter 21, at an electrogram acquisition step 302. Next, processor 41 applies an algorithm to detect, by one of the methods described in FIGS. 4 and 5 below, or other methods, if the acquired electrogram suffers from the presence of injury current, at an injury current detection step 304.

At a checking step 306, the processor 41 checks if it has detected an injury current. If the answer is “yes,” the processor drops the electrogram from use in an EP map, at a signal dropping step 308. If the answer is “no,” the processor uses the electrogram in an EP map, at a signal using step 310. Using the electrogram can mean annotating an LAT value on the electrogram to include a data point in an LAT map.

### Methods to Detect Presence of Injury Current in Cardiac Electrophysiological Signal

FIG. 4 is a flow chart describing a method for detecting electrogram distorted by injury current by comparing electrogram peak amplitude to threshold, and dropping such electrograms, according to an embodiment of the present invention. The algorithm, according to the presented embodiment, carries out a process that begins with interface circuits 38 receiving a unipolar electrogram acquired by catheter 21, such as electrogram 202, at a unipolar electrogram acquisition step 402. Next, processor 41 applies an algorithm to detect, by comparing peak electrogram signal at a given window of interest (WOI) to threshold 206, if the acquired electrogram suffers from presence of injury current, at an injury current detection step 404.

At a checking step 406, the processor 41 checks if threshold 206 has been exceeded. If the answer is “yes,” the processor drops the unipolar electrogram from use in EP map, at a signal dropping step 408. If the answer is “no,” the processor uses the unipolar electrogram in EP map, at a signal using step 410. Using the electrogram can mean annotating an LAT value on the electrogram and include that data point in an LAT map and/or in a potential map.

FIG. 5 is a flow chart describing a method for detecting an electrogram distorted by injury current by comparing baseline signals, according to another embodiment of the present invention. The algorithm, according to the presented embodiment, carries out a process that begins with interface circuits 38 receiving an EP signal from central reference electrode 58 relative to the WCT terminal described in FIG. 1, at a reference signal acquisition step 502. Next, processor 41 applies an algorithm to measure a baseline potential component V_{CRE} of the signal, at a reference baseline signal measurement step 504.

In parallel, interface circuits 38 receive an EP signal from a spine electrode 48 relative to the WCT terminal, at a tissue signal acquisition step 506. Next, processor 41 applies an algorithm to measure a baseline potential component Vse of the tissue signal, at a tissue baseline signal measurement step 508.

At a baseline signal comparison step 510, processor 41 checks if the two baseline signals are equal (up to a given tolerance). If the answer is “no,” the processor indicates (512) that a respective unipolar electrogram signal between electrodes 48 and 58 suffers from a certain level of injury current. If the answer is “yes,” the processor indicates (514) that no injury current was detected therein.

The processor may use or drop the respective unipolar electrogram, based, for example, on the condition of step 410, or by comparison (V_{SE}−V_{CRE}) to a threshold value.

It will be appreciated that the embodiments described above are cited by way of example, and that the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A system comprising:
a catheter comprising:
an elongated shaft extending along a longitudinal axis;
an expandable assembly disposed at a distal end of the elongated shaft and comprising a plurality of spines;
a sensing electrode coupled to at least one spine of the plurality of spines; and
a reference electrode; and
one or more processors configured to receive one or more signals from the sensing electrode and the reference electrode, the one or more processors configured to compare the one or more signals to a predefined threshold to detect a presence of an injury current.

2. The system of claim 1, wherein the one or more signals comprises a unipolar signal acquired between the reference electrode and the sensing electrode.

3. The system of claim 2, wherein the one or more processors are configured to estimate a level of injury current by comparing a peak level of the unipolar signal to the predefined threshold.

4. The system of claim 3, wherein the one or more processors are configured to use the one or more signals to construct an electrophysiological map when the level of injury current is below the predefined threshold, and to discard the one or more signals when the level of injury current is above the predefined threshold.

5. The system of claim 2, wherein the reference electrode is positioned at a center of the plurality of spines.

6. The system of claim 2, wherein the expandable assembly is a basket assembly.

7. The system of claim 2, wherein the reference electrode is immersed in blood and the sensing electrode is in contact with tissue when the unipolar signal is acquired.

8. The system of claim 1, further comprising a surface electrode, and wherein the one or more signals comprises a first signal between the sensing electrode and the surface electrode and a second signal between the reference electrode and the surface electrode.

9. The system of claim 8, wherein the one or more processors are configured to indicate the presence of the injury current if a difference between the first signal and the second signal exceeds the predefined threshold.

10. The system of claim 9, wherein the one or more processors are configured to:
receive a unipolar signal between the sensing electrode and the reference electrode; and
use the unipolar signal to construct an electrophysiological map when the difference between the first signal and the second signal is below the predefined threshold; or
discard the unipolar signal when the difference between the first signal and the second signal is above the predefined threshold.

11. The system of claim 10, wherein the reference electrode is immersed in blood and the sensing electrode is in contact with tissue when the unipolar signal is acquired.

12. The system of claim 9, wherein the difference between the first signal and the second signal is acquired by comparing a baseline of the first signal to a baseline of the second signal.

13. The system of claim 8, wherein the reference electrode is positioned at a center of the plurality of spines.

14. The system of claim 8, wherein the reference electrode is positioned at a center of the expandable assembly.

15. A method, comprising:
positioning an expandable assembly of a catheter into a heart of a patient, the expandable assembly comprising:
a plurality of spines disposed at a distal end of an elongated shaft, the elongated shaft extending along a longitudinal axis;
a sensing electrode coupled at least one of the spines; and
a reference electrode;
positioning a surface electrode on a skin surface of the patient;
receiving a first signal, the first signal comprising a first electrical signal transmitted between the sensing electrode and the surface electrode;
receiving a second signal, the second signal comprising a second electrical signal transmitted between the reference electrode to the surface electrode;
comparing the first signal and the second signal; and
indicating a presence of an injury current if a difference between the first signal and the second signal exceeds a predefined threshold.

16. The method of claim 15, wherein positioning the expandable assembly into the heart of the patient comprises contacting tissue with the sensing electrode and immersing the reference electrode in blood.

17. The method of claim 15, further comprising:
receiving a unipolar signal between the sensing electrode and the reference electrode; and
using the unipolar signal to construct an electrophysiological map when the difference between the first signal and the second signal is below the predefined threshold; or
discarding the unipolar signal when the difference between the first signal and the second signal is above the predefined threshold.

18. The method of claim 15, wherein comparing the first signal to the second signal comprises comparing a baseline of the first signal to a baseline of the second signal.

19. A method, comprising:
positioning an expandable assembly of a catheter into a heart of a patient, the expandable assembly comprising:
a plurality of spines disposed at a distal end of an elongated shaft, the elongated shaft extending along a longitudinal axis;
a sensing electrode coupled at least one of the spines; and
a reference electrode;
receiving a signal comprising an electrical signal transmitted between the sensing electrode and the reference electrode;
comparing a peak of the signal to a predefined threshold; and
indicating a presence of an injury current if the peak of the signal is greater than the predefined threshold.

20. The method of claim 19, further comprising:
using the signal to construct an electrophysiological map when the peak of the signal is below the predefined threshold; or
discarding the signal when the peak of the signal is above the predefined threshold.
